(19) United States
(12) Patent Application Publication
(10) Pub. No.: US 2011/0142819 A1
(43) Pub. Date: Jun. 16, 2011

(54) METHOD OF IMPROVING WOULD HEALING

(75) Inventors: Tor Ny, Umea (SE); Jinan Li, Stockholm (SE); Sten Hellstrom, Limea (SE); Per-Olof Eriksson, Umea (SE)

(73) Assignee: Omnio Healer AB, Umea (SE)

(21) Appl. No.: 12/863,161

(22) PCT Filed: Jan. 21, 2009
(86) PCT No.: PCT/SE2009/050057

§ 371 Date: Feb. 22, 2011

Related U.S. Application Data
(60) Provisional application No. 61/022,533, filed on Jan. 22, 2008.

(51) Int. Cl. A61K 38/48; A61K 38/14; A61P 17/02

(52) U.S. Cl. 424/94.64; 514/20.9; 514/18.6

(57) ABSTRACT

This invention relates to novel methods of promoting the healing or closure of perforated tympanic membranes or wounds, as well as methods for minimizing scar formation and removing necrotic tissue. In particular the invention relates to the use of components of the plasminogen activating system, especially mini-plasminogen, mini-plasmin, micro-plasminogen, micro-plasmin, delta-plasminogen, and delta-plasmintopromote wound healing processes.

### FIELD OF THE INVENTION

This invention relates to wound healing processes. In particular, the invention relates to novel methods of promoting the healing or closure of perforated tympanic membranes or wounds, as well as methods for minimizing scar formation and removing necrotic tissue. The invention also relates to animal models for studying wound-healing processes, and screening methods for identifying and evaluating drugs and treatment methods for enhancing healing of perforated tympanic membranes or wounds.

### BACKGROUND

Slow or improper wound healing compromises the quality of life for large numbers of people. One particular type of wound healing where problems can occur is the healing of tympanic membrane (eardrum) perforations. Although the majority of perforations will heal spontaneously, and are closed by proliferating keratinizing squamous epithelium advancing ahead of an ingrowing connective tissue, some perforations do not heal, frequently resulting in loss of hearing or other complications. It is still an open question why some perforations heal, whereas others stay patent. In addition, every year in the United States more than 1.25 million people suffer from burns, and 6.5 million have chronic skin ulcers caused by pressure, venous stasis, or diabetes mellitus.

Wound healing is a dynamic tissue remodeling process involving the formation of a matrix rich in fibrin and fibronectin in the wound field, infiltration of neutrophils and macrophages, proliferation of epidermal keratinocytes at the wound edges and their migration through the provisional matrix, formation of granulation tissue containing newly developed vessels and migrating inflammatory cells and fibroblasts, and wound contraction. Wound healing studies of skin suggest that proteases play important roles in several steps. It is well documented that the degradation of the extracellular matrix (ECM) that takes place during wound healing and other ECM remodeling processes is dependent on the action of a variety of proteolytic enzymes secreted by inflammatory cells, as well as by stromal tissue cellular elements. Many different proteinases are thought to contribute to matrix remodeling during wound healing (Saksela and Rifkin, Annu. Rev. Cell Biol. 4, 93-126 (1988)). However, the precise mechanisms responsible for this process, and how they are regulated, are poorly understood.

### The Plasminogen-Activation System

The plasminogen-activation system is a versatile, temporally controlled enzymatic system in which plasminogen is activated to the proteolytic enzyme plasmin by either of the two physiological plasminogen activators (PAs), tissue-type plasminogen activator (tPA) and urokinase-type plasminogen activator (uPA). Activation of this system is initiated by the release of tPA or uPA by specific cells in response to external signals and leads to a locally expressed extracellular proteolytic activity (Vassalli et al. J. Exp. Med. 159, 1653-1668 (1984); Saksela & Rifkin, 1988, supra). The PA-system is also regulated by specific inhibitors directed against PAs and plasmin, including PA-inhibitor type 1 (PAI-1), PA-inhibitor type 2 (PAI-2), protease nexin 1 (PN-1) and α2-anti-plasmin (Saksela & Rifkin, 1988, supra; Ny et al., Thromb Res. 71(1):1-45 (1993)). All of these inhibitors, which belong to the serpin family, are suicide inhibitors that are cleaved by cognate protease (Wilczynska et al., J Biol. Chem. 270(50):29652-5 (1995); Wilczynska et al., Nat Struct Biol. 4(5):354-7 (1997)). The most important feature of the PA-system is the amplification achieved by the conversion of plasminogen resulting in the formation of plasmin.

PAs have been found to be present in wound edges, along with several types of matrix metalloproteinases (MMPs), including interstitial collagenase (MMP-1), stromelysin-1 (MMP-3), and the latent forms of gelatinase A (MMP-2) and gelatinase-B (MMP-9). The expression of both PAs and MMPs is induced by inflammatory mediators and cytokines, indicating that the two enzyme systems may act in concert. MMPs are known to be synthesized as latent precursor enzymes that can be activated by limited proteolysis, but the exact mechanism by which this activation takes place in vivo is largely unknown. Plasmin is one of the factors proposed to be involved in the activation of some subclasses of metalloproteinases (Lijnen, Thromb Haemost 86(1):324-33 (2001)).

A number of reports have indicated that the expression or activation of MMPs, tissue inhibitors of metalloproteinases (TIMPs), PAs and PA-inhibitors are altered in wound healing processes, and there have also been indications that plasmin plays a role in skin wound healing (Romer et al., Nat. Med. 2:287-292 (1996)). Moreover, U.S. Pat. Nos. 5,925,350 and 6,03,664 to Verheijen describe the use of uPA or tPA to improve wound healing of slow- or non-healing wounds. In these studies, it was noted that the improvement mechanism was not associated with fibrinolytic activity or necrotic tissue removal. Specific strategies for improving tympanic membrane healing have also been proposed, using topical application of high concentrations of hyaluronan (Laurent et al., Arch Otolaryngol Head Neck Surg 114:1435-1441 (1988)) or basic fibroblast growth factor (bFGF; Fina et al., Laryngoscope 103(7):804-809 (1993)).

While the understanding of wound healing and the various mechanisms regulating it is improving, and promising treatment strategies have been proposed, the healing of slow- or non-healing tympanic membrane perforations or other wounds remain a medical as well as social problem. There is thus a need in the art for new and improved methods for accelerating wound healing processes, such as healing of tympanic membrane perforations, burns and skin ulcers, removing any necrotic tissue, and minimizing scar formation. There is also a need for new screening methods by which to identify and evaluate drugs that can be used in such treatment methods. The invention addresses these and other needs in the art.

### SUMMARY OF THE INVENTION

The present invention provides a new method for improving the healing of tympanic membrane perforations, or minimizing scar formation during healing, by administration of an agent that is a component of the plasminogen activating system. Preferably, the agent is selected from plasmin or plasminogen or an analogue of plasmin or plasminogen. Most preferably, the agent is micro-plasminogen, micro-plasmin, mini-plasminogen, mini-plasmin, delta-plasminogen, or delta-plasmin.

Accordingly, the invention provides a method of promoting healing of a tympanic membrane perforation in a subject in need of such treatment, which comprises administering to the subject a composition containing an effective amount of a component of the plasminogen activating system for promoting healing of the tympanic membrane perforation. Preferably, the subject is a human, and the component of the plasminogen activating system is a component of the human plasminogen activating system. The composition may further comprise a pharmaceutically acceptable carrier, and can be in the form of an aqueous solution, a gel, a lotion, a balm, a powder, a paste, a bandage, a wound dressing, or another suitable delivery vehicle. The component of the plasminogen activating system can be administered topically or systemically. In the case of topical administration, the administered composition may comprise from about 0.05 mg/ml to about 50 mg/ml of the component of the plasminogen activating system, preferably from about 015 mg/ml to about 10 mg/ml of the component of the plasminogen activating system, or even more preferably from about 0.5 mg/ml to about 5 mg/ml of the component of the plasminogen activating system. The composition may promote healing by accelerating the healing of the perforation, reducing necrotic tissue, and reducing the formation of scar tissue in the wound area. In one embodiment, the administration of the component of the plasminogen activating system is repeated at least once, preferably at least once every day.

The invention also provides a method of reducing scar formation from a healing wound in a subject in need of such treatment, which comprises administering to the subject a composition containing an effective amount of a component of the plasminogen activating system for reducing scar formation. The component of the plasminogen activating system can, for example, reduce fibrin deposition. The subject is preferably a human subject, and the component of the plasminogen activating system is preferably a component of the human plasminogen activating system. In one embodiment, the component of the plasminogen activating system is administered topically, and the component of the plasminogen activating system is administered so that from about 0.5 mg to about 5 mg component of the plasminogen activating system is applied per square centimeter wound area.

The invention also provides for a method of accelerating wound healing in a patient in need of such treatment, which comprises administering to the patient a composition containing an effective amount of a component of the plasminogen activating system to promote healing of the wound. Optionally, the wound is a chronic wound. The subject may be a human subject, in which case the component of the plasminogen activating system is preferably, although not necessarily, a component of the human plasminogen activating system. In case the component of the plasminogen activating system is administered topically, it may be administered so that from about 0.5 mg to about 5 mg component of the plasminogen activating system is applied per square centimeter wound area.

The invention also provides for a method of reducing necrotic tissue formation in a healing wound in a subject in need of such treatment, which comprises administering to the subject a composition containing an effective amount of a component of the plasminogen activating system for reducing necrotic tissue formation. Optionally, the component of the plasminogen activating system reduces fibrin deposition. In one embodiment, the subject is a human subject, and the component of the plasminogen activating system is a component of the human plasminogen activating system. If administered topically, the composition may be applied in an amount corresponding to, for example, from about 0.5 mg to about 5 mg component of the plasminogen activating system per square centimeter wound area.

The above features and many other advantages of the invention will become better understood by reference to the following detailed description when taken in conjunction with the accompanying drawings.

### BRIEF DESCRIPTION OF THE DRAWINGS

FIG. 1. Topical application of plasminogen on perforated tympanic membranes. Time course of (A) inflammatory reaction in the mid-ear cavity and (B) shrinkage of perforation, after tympanic membrane perforation in wild-type rats. Fifty μl plasminogen (50 μg (1 mg/ml)----▪----; or 0.5 mg (10 mg/ml)---▴---) or control (PBS, ---▾---) solution was administered after perforation, and thereafter every 24 hours.

FIG. 2. Healing acceleration effect of p1g in burn wounds in rats. A. Quantatitive analysis of burn wound size in rats after plasminogen or control PBS treatments. B. Quantatitive analysis of the duration of scabs before falling off in rats treated with plasminogen or control PBS.

FIG. 3. Quantitative analysis of the incisional wound areas in rats treated with control PBS or p1g. Wound area at day 17, 8 days after local treatment of rats with plasminogen or control PBS.

### DETAILED DESCRIPTION OF THE INVENTION

The present invention relates to healing of perforated tympanic membranes, and to healing of wounds. The invention is also applicable to diseases and conditions characterized by degeneration or poor healing of extracellular matrix structures, particularly the keratinized tissue such as, e.g., the tympanic membrane. Other abnormal wound healing processes include diabetic ulcers, keloids, hypertrophic scars, and the application of skin substitutes.

The invention is based, in part, on the discovery that in the absence of plasminogen, the wound healing process does not progress properly, showing that plasminogen plays a pivotal role in the healing of wounds, particularly in the case of tympanic membrane perforations (see Examples and Table 1). As shown by the Examples, healing of tympanic membrane perforations was dramatically altered and abnormal in mice lacking plasminogen as compared to wild-type controls and other transgenic models (see Table 1). In plasminogen deficient mice, tympanic membrane perforations did not heal properly during the 144-day test period during which healing was monitored, resulting in an abnormal tympanic membrane. It was also found that plasminogen-deficient mice reverted to a normal healing process following injection of human plasminogen. (see Examples 2 and 5), and that wild-type rats displayed enhanced tympanic membrane healing after administration of plasminogen (Example 6). In addition, experiments showed that uPA, but not tPA, may also play some role in normal wound healing, in that uPA appeared to be needed for the removal of fibrin depositions (Example 3 and 4). However, in no animal model was the lack of normal healing more apparent than in plasminogen-deficient mice, indicating that plasminogen plays a more essential and probably different role during wound healing than uPA. Without being bound to any specific theory, the present findings supports that the inability to proper healing may be caused by a defect in the inflammatory response, keratinocyte migration, or matrix remodeling events during the healing process.

As demonstrated by the results in Table 1, uPA, tPA, and plasminogen play distinct roles in the wound healing process, with plasminogen playing a pivotal role. In addition, as shown in the Examples, removal of necrotic tissue and fibrin are critically dependent on the presence or administration of plasminogen/plasmin rather than tPA or uPA.

TABLE 1
Comparison of Time of Healing and Appearance of Healed Tympanic Membrane
Tissue in Wild-Type, uPA-Deficient, tPA-Deficient, and Plasminogen-Deficient Mice

| Genotype | Day of healing | Healing Pattern | Necrosis | Fibrin deposition |
|---|---|---|---|---|
| Wild-type | 8.7 ± 2.4 | Normal healing | No | No |
| tPA^{-/-} | 9.0 ± 2.4 | Apparently normal | No | No |
| | | healing | | |
| uPA^{-/-} | 11.0 ± 1.7 | Healed area contains | No | Spots of fibrin |
| | (7/8 mice healed | ruffled surface and | | depositions at healed |
| | at day 11) | cobble-stone-like tissue | | area. |
| | | deposits | | |
| Plg^{-/-} | — | Tympanic membrane | Necrosis | Extensive fibrin |
| | (0/8 mice healed | covered by whitish tissue, | becomes | deposition that |
| | at day 144) | and is thick and non- | apparent from | completely covers |
| | | transparent | day 16 and | tympanic membrane |
| | | | onwards | surface |

Accordingly, a component of the plasminogen activating system can be used in the treatment of wound healing diseases, as well as for acceleration strategies for tympanic membrane and wound healing, treatment of conditions or diseases affecting healing of disrupted epidermal tissues, methods for reducing scar formation or necrotic tissue accumulation or formation, as well as screening methods for drugs to be used in such treatments. The administration of a component of the plasminogen activating system can also minimize scar formation or necrotic tissue accumulation in tympanic membranes or other epidermal tissues during wound healing. For example, a component of the plasminogen activating system can be applied in conjunction with plastic surgery to reduce the appearance of, or prevent, the formation of residual scars, fibrin deposits, or necrotic tissue. Also, component of the plasminogen activating system can be applied onto ulcers or burns to improve healing.

In addition, the method of the invention can be used for improving wound healing in conditions of local or systemic deficiency of plasminogen, or to improve the healing of non- or slow-healing wounds. Notably, as shown in Example 5, restoration of plasminogen weeks after injury can diminish the accumulated extracellular matrix and restart normal healing, thus showing that plasminogen can be applied for treatment of chronic wounds such as ulcerations and bedsores.

The compositions of the invention can be administered topically, by injection, or by intravenous infusion. Preferably, although not necessarily, the administration is local, i.e., in some proximity to the wound. When the component of the plasminogen activating system is administered by injection (e.g., intravenously, subcutaneously, intramuscularly), it is advantageously prepared as a solution of the material in a pharmaceutically acceptable liquid, such as, e.g., isotonic saline. In a preferred embodiment, the component of the plasminogen activating system is administered locally to attain a high concentration, for example, at least 100 μg/ml of component of the plasminogen activating system plasminogen, in the perforation or wound area. For topical administration, the component of the plasminogen activating system composition may, for example, be part of a gel, lotion, balm, paste, spray, powder, bandage, or wound dressing. For accelerating healing of a tympanic membrane perforation, the composition is preferably administered via the outer ear canal, by, e.g., a spray delivered to the area of the perforation, or by adding the component of the plasminogen activating system solution drop-wise. Devices for delivering compositions by spray are known in the art, and is described in, e.g., U.S. Pat. No. 6,027,712. Strategies such as administering the component of the plasminogen activating system by spray, as well as by gel or paste, or a topically administered solution, can also be used for treating wounds in the oral cavity. For acceleration of wound healing, the component of the plasminogen activating system can be present in a wound dressing applied onto the wound, from which it is transferred to the wound area.

### DEFINITIONS

The terms used in this specification generally have their ordinary meanings in the art, within the context of this invention and in the specific context where each term is used. Certain terms are discussed below, or elsewhere in the specification, to provide additional guidance to the practitioner in describing the compositions and methods of the invention and how to make and use them.

“Tympanic membrane” and “eardrum” are used interchangeably herein.

A “tympanic membrane perforation” is an opening in the tympanic membrane usually caused by trauma. There are at least four general categories: compression injuries (the most common and usually the result of a blow to the ear); instrumentation injuries (the second most common, usually inadvertent, caused often by cotton swabs or bobby-pins); burn-slag injuries (frequently seen in industry, from hot metal from machines or welding); and blast injuries (usually seen during war or bombings). Infection can cause delayed healing of the tympanic membrane, and persistent perforation is usually a manifestation of tubotympanitis, an inflammation of the eustachian tube and tympanic cavity (middle ear).

A “wound” is a break or discontinuity in the structure of an organ or tissue, including epithelium, connective tissue, and muscle tissue, caused by an external agent. Examples of wounds include, but are not limited to, skin wounds, bruises, ulcerations, bedsores, grazes, tears, cuts, punctures, psoriasis wounds, tympanic membrane perforations, and burns. Particular types of wounds are those that are a consequence of plastic surgery procedures.

“Topical” and “topical application” refer to non-systemic, local, administration of an active ingredient. Thus, topical application can refer to application of an active ingredient to the external surface of a wound.

A “component of the plasminogen activation pathway” is a compound having the final effect of activating plasminogen, e.g. by a cascade of events resulting in the formation or activation of plasminogen. Examples of such compounds are plasmin or plasminogen or an analogue of plasmin or plasminogen, micro-plasminogen, micro-plasmin, mini-plasminogen, mini-plasmin, delta-plasminogen, or delta-plasmin, PA activators, especially uPA, tPA and other types of activator homologues like streptokinase. An “analogue” of plasminogen or plasmin is a compound providing essentially an analogous effect as plasminogen or plasmin, respectively, as measured by enzymography, ELISA (enzyme-linked immunosorbent assay) and FACS (fluorescence activated cell sorter), There is also an assay for measuring levels of converted plasmin activity as described previously: Ny, A., Leonardsson, G., Hagglund, A. C., Hagglof, P., Ploplis, V. A., Carmeliet, P., and Ny, T. (1999). Ovulation in plasminogen-deficient mice. Endocrinology 140, 5030-5035.). An “analogue” of a component of the plasminogen activation pathway is a compound providing essentially an analogous effect as a component of the plasminogen activation pathway as measured by the levels of plasmin activity that this analogue activates.

“Plasminogen” herein includes endogenously occurring mammalian plasminogen, allelic plasminogen, function-conservative derivatives of plasminogen, functionally active plasminogen fragments, and mammalian plasminogen homologs. Optionally, a plasminogen composition may contain more than one type, derivative, or homolog of plasminogen. Preferably, the type of plasminogen to be used in a composition to be administered to a subject is endogenous to the species of the subject. A preferred compound is plasminogen purified from a biological source, e.g., recombinantly produced human plasminogen, or purified human plasminogen, which is available from, for example, Biopool AB (Umeå, Sweden). One preferred human plasminogen has the amino acid sequence of GenBank Accession No. PLHU (GI:625234).

“Plasmin” herein includes endogenously occurring mammalian plasmin, allelic plasmin, function-conservative derivatives of plasmin, functionally active plasmin fragments, and mammalian plasmin homologs. Optionally, a plasmin composition may contain more than one type, derivative, or homolog of plasmin. Preferably, the type of plasmin to be used in a composition to be administered to a subject is endogenous to the species of the subject. A preferred compound is plasmin purified from a biological source, e.g., recombinantly produced human plasmin, or purified human plasmin.

“Mini-plasminogen” and “mini-plasmin” are plasminogen and plasmin derivatives which can be prepared by protelytic digestion by elastase from plasminogen and plasmin, respectively. Mini-plasminogen and mini-plasmin are described by Sottrup-Jensen et al. (Progr Chem Fibrinol Thrombol (Davidson et al. eds) 1978, Vol 3:191-209, Raven Press).

“Mikro-plasminogen” and mikro-plasmin” are plasminogen and plasmin derivatives produced by the action of plasmin on plasminogen and plasmin, respectively. Mikro-plasminogen and mikro-plasmin are described in WO 89/01336 and the corresponding U.S. Pat. No. 4,774,087, the contents of which hereby are incorporated by reference.

“Delta-plasminogen” and “delta-plasmin” refers to modified plasmin and plasminogen molecules having a single N-terminal kringle domain and a C-terminal domain activation site and serine protease domain. Delta-plasminogen and delta-plasmin are described in WO 2005/105990 and the corresponding U.S. application 60/564,472 the contents of which hereby are incorporated by reference.

“Function-conservative variants” are proteins in which a given amino acid residue has been changed without altering overall conformation and function of the protein, including, but not limited to, replacement of an amino acid with one having similar properties (such as, for example, acidic, basic, hydrophobic, and the like). Amino acids with similar properties are well known in the art. For example, arginine, histidine and lysine are hydrophilic-basic amino acids and may be interchangeable. Similarly, isoleucine, a hydrophobic amino acid, may be replaced with leucine, methionine or valine Amino acids other than those indicated as conserved may differ in a protein or enzyme so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A “function-conservative variant” also includes a polypeptide or enzyme which has at least 60% amino acid identity as determined by BLAST or FASTA algorithms, preferably at least 75%, more preferably at least 85%, even more preferably at least 90%, and still more preferably 95%, and which has the same or substantially similar properties or functions as the native or parent protein or enzyme to which it is compared.

A “subject” herein includes both human and non-human animals. Non-human animals include, without limitation, mammals, laboratory animals such as mice, rats, rabbits, hamsters, guinea pigs, etc.; domestic animals such as dogs and cats; and farm animals such as sheep, goats, pigs, horses, and cows. A non-human animal of the present invention may be a mammalian or non-mammalian animal; a vertebrate or an invertebrate.

“Treatment” of a subject, or “treating” a subject for a disease or condition herein means reducing or alleviating clinical symptoms of the disease or condition such as impaired or slow wound-healing.

“Promote”, “enhance”, or “improve” tympanic membrane or wound healing generally means increasing the speed by which the wound or perforation heals or reducing the extent of residual scar or necrotic tissue during or after healing of the wound or perforation.

A “control”, “control value” or “reference value” in an assay is a value used to detect an alteration in, e.g., the healing of a perforated tympanic membrane or skin wound, or any other assays described herein. For instance, when studying healing of a tympanic membrane perforation, the inhibitory/stimulatory effect of an agent can be evaluated by comparing the healing of a wound or perforation to that of a control. The control or reference may be, e.g., a predetermined reference value, or may be determined experimentally. For example, in such an assay, a control or reference may be the healing of a similar wound or perforation in an animal not exposed to the drug or active agent, or an animal treated with the same drug or active agent which does not have impaired wound healing capability

An “effective amount” or a “therapeutically effective amount” means an amount that increases the local and/or systemic concentrations of the component of the plasminogen activating system, and/or enhances wound healing. For example, an effective amount of an active agent can be an amount that results in a local (in the perforation, wound, or scar area) or systemic level of the component of the plasminogen activating system that exceeds 200 microgram/ml. Alternatively, an effective amount of an agent is an amount that results in a faster healing of a perforation or wound than in the absence of the agent, or a reduced scar or necrotic tissue formation than in the absence of the agent. An effective amount could also mean an amount or dose sufficient to increase the local and/or systemic levels of plasminogen, e.g., to about 10 percent, preferably by about 50 percent, and more preferably by about 100 percent of the level found before administration of the active agent or drug. Alternatively, an effective amount of the component of the plasminogen activating system is an amount corresponding to about 5 μg to about 50 mg, preferably about 0.05 mg to about 10 mg, and, even more preferably, from about 0.5 mg to about 5 mg component of the plasminogen activating system per square centimeter in the wound area. A therapeutically effective amount can ameliorate or present a clinically significant response in a subject, in that, e.g., tympanic membrane perforation or wound healing is promoted, or scar formation reduced. Alternatively, a therapeutically effective amount is sufficient to improve a clinically significant wound-healing or scar formation condition in the host.

As used herein, “about” or “approximately” shall mean within 50 percent, preferably within 20 percent, more preferably within 5 percent, of a given value or range.

A value which is “substantially different” from another value can mean that there is a statistically significant difference between the two values. Any suitable statistical method known in the art can be used to evaluate whether differences are significant or not. A “statistically significant” difference means a significance which is determined at a confidence interval of at least 90%, more preferably at a 95% confidence interval.

In accordance with the present invention there may be employed conventional molecular biology, microbiology, and recombinant DNA techniques within the skill of the art. Such techniques are explained fully in the literature. See, e.g., Sambrook et al. (Molecular Cloning-A Laboratory Manual, Cold Spring Harbor Laboratory Press, 1989); Glover (DNA Cloning: A Practical Approach, Volumes I and II, 1985); Hames and Higgins (Nucleic Acid Hybridization, 1985); Hames and Higgins (Transcription And Translation, 1984); Freshney (Animal Cell Culture, 1986); Perbal (A Practical Guide To Molecular Cloning, 1984); and Ausubel et al. (Current Protocols in Molecular Biology, John Wiley & Sons, Inc., 1994).

Abbreviations used in the present disclosure include the following:

uPA=Urokinase-type plasminogen activator;

PA=Plasminogen activator;

MMP=Matrix metalloproteinase;

TIMP=Tissue inhibitor of metalloproteinase;

tPA=Tissue-type plasminogen activator;

Plg=Plasminogen

ECM=Extracellular matrix

### Improvement of Wound Healing

According to the invention, wound healing is improved by providing or enhancing the levels of a component of the plasminogen activating system. This may be accomplished in several different ways. For instance, a patient may be treated with an effective amount of an active agent such as, e.g., a drug, hormone, cytokine, antibody, or another compound that up-regulates the expression of plasminogen; reduces the degradation of plasminogen; or increases the local or systemic levels of plasminogen or a plasminogen homolog or derivative. Nucleic acids encoding plasminogen may also be administered for therapeutic purposes.

In a preferred embodiment, a substantially pure preparation of a component of the human plasminogen activating system is used. The component of the plasminogen activating system may be produced by purifying the component from humans or other animals, or by recombinant production in host cells, including prokaryotic host cells such as S. cerevisiae or E. coli, and, more preferably, mammalian host cells such as CHO cells. In particular, fragments of the component which retain at least a part of the desired activity of the full-length component may be used

### Applications

The method of the invention may be used to accelerate the healing of a tympanic membrane or other wound in animals including, but not limited to, vertebrates such as humans and domestic animals, including dogs, cats, and horses. In addition, plasminogen may be used in clinical applications to reduce scar tissue formation and necrotic tissue in a wound area, and to enhance removal of tissue debris.

In one embodiment, the method of the invention is applied for enhancing the healing of a perforated tympanic membrane in a human subject. The human or non-human subject may or may not suffer from a condition which impairs or slows down the healing of the tympanic membrane perforation.

In another embodiment, the method of the invention provides a method for accelerating the healing of a wound in a subject. The subject may or may not suffer from a condition which is associated with the appearance of wounds, or from a condition that affects wound healing, such as diabetes (e.g., diabetic ulcer keloids), chronic wounds such as ulcers or bedsores.

Since the method of the invention not only increases the wound healing rate, but also improves the quality of the wounds, i.e., reduces the appearance of scars and the appearance of necrotic tissue, a composition which enhances the levels of a component of the plasminogen activating system at least in the wound area can be administered to any patient to reduce scar formation. In a particular embodiment, the subject is a human which plans to undergo, is undergoing, or has undergone, plastic surgery or application of a skin substitute. In such a case, a composition comprising the component of the plasminogen activating system can be applied or administered both prior to and/or after surgery.

Furthermore, the administration of the component of the plasminogen activating system can restart a normal wound healing pattern in situations of impaired wound healing. Without being bound to any particular theory, besides fibrinolysis, elevated plasminogen levels may lead to increased plasmin, which may initiate a wound healing process through activation of cytokine pathways.

### Compositions and Treatments

The invention provides composition comprising a component of the plasminogen activating system, which, when administered in an effective amount, results in an increased level of a component of the plasminogen activating system in the wound area of a subject and thereby improved healing of a tympanic membrane perforation or other wound.

For example, for acceleration of tympanic membrane healing, a composition comprising about 5 μg to about 50 mg, preferably about 0.05 mg to about 10 mg, and, even more preferably, from about 0.5 mg to about 5 mg of the component of the plasminogen activating system, can be applied to the area surrounding a tympanic membrane perforation. Alternatively, about 10-50 μl of a 1 mg/ml solution of the component of the plasminogen activating system, or the corresponding amount delivered in another suitable formulation, can be applied. For acceleration of the healing of wounds in general, a composition comprising a component of the plasminogen activating system can be applied so that the amount of the component of the plasminogen activating system per each square centimeter (cm²) of the wound area comprises about 5 μg to about 50 mg, preferably about 0.05 mg to about 10 mg, and, even more preferably, from about 0.5 mg to about 5 mg of the component of the plasminogen activating system.

Alternatively, the component of the plasminogen activating system is administered so that a locally high concentration of the component of the plasminogen activating system is achieved in the tympanic membrane perforation or wound area of a subject, e.g., a human patient. In a preferred embodiment, the administration of an effective amount of the active agent results in a concentration of the component of the plasminogen activating system of at least about 20 μg/ml in the perforation or wound area. In yet another preferred embodiment, the administration of an effective amount of the active agent results in a concentration of the component of the plasminogen activating system of at least about 200 μg/ml in the perforation or wound area. In still another preferred embodiment, the concentration of the component of the plasminogen activating system resulting from administration of an effective amount of an active agent is from about 2 μg/ml up to about 2 mg/ml, more preferably at least 200 μg/ml. The concentration of plasminogen in human plasma is about 200 microgram per ml.

Accordingly, the component of the plasminogen activating system may be formulated into pharmaceutical compositions for administration to subjects in a biologically compatible form suitable for administration in vivo, e.g., by topical administration, injection, or infusion. By biologically compatible form suitable for administration in vivo is meant a form of the substance to be administered in which any toxic effects are outweighed by the therapeutic effects. Administration of a therapeutically active amount of the pharmaceutical compositions of the present invention is defined as an amount effective, at dosages and for periods of time necessary to achieve the desired result. For example, a therapeutically active amount of a substance may vary according to factors such as the disease state, age, sex, and weight of the individual. Dosage regima may be adjusted to provide the optimum therapeutic response. For example, more than one divided doses may be administered daily or the dose may be proportionally reduced as indicated by the exigencies of the therapeutic situation.

If desired, more than one component of the plasminogen activating system may also be applied for treatment of a perforation or wound.

The component of the plasminogen activating system may be administered in a convenient manner such as by injection (subcutaneous, intravenous, etc.), inhalation, spray, topical or transdermal application, or rectal administration. Depending on the route of administration, the active substance may be coated in a material to protect the compound from the action of enzymes, acids and other natural conditions that may inactivate the compound. Thus, suitable routes of administration include topical, intravenous, intramuscular, intradermal, rectal, and intravaginal administration. A preferred administration route is topical administration. In the case of tympanic membrane perforations, the active agent can be administered via the outer ear canal. The vessels that support the tympanic membrane stop at the border of the membrane. Accordingly, blood and blood components such as oxygen, nutrition and plasminogen only reach the tympanic membrane by diffusion.

The compositions described herein can be prepared by methods known per se for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (Mack Publishing Company, Easton, Pa., USA 1985). On this basis, the compositions include, albeit not exclusively, solutions of the substances or compounds in association with one or more pharmaceutically acceptable vehicles or diluents, and contained in buffered solutions with a suitable pH and iso-osmotic with the physiological fluids.

Examples of vehicles that may be used in delivering the component of the plasminogen activating system include, but are not limited to, injectable dosage forms, infusions, gel, pastes, balms, waxes, lotions, skin creams, and various other formats for topical administration known in the art. The compositions may also be delivered locally in the form of a powder or solution sprayed onto the wound area. Alternatively, the compositions of the invention may be present in wound dressings, pads, band-aids, gauze, or other means applied onto a wound, from which they are transferred to the wound area. Such devices also include slow-release devices, continually releasing plasminogen or other components for a prolonged period of time. With respect to healing of tympanic membranes, administration of the composition using a gel, spray, or drop-wise application via the outer ear canal, is one preferred embodiment. Injectable dosage forms or infusions comprise a solution of the component of the plasminogen activating system in a pharmaceutically acceptable liquid such as, e.g., isotonic saline, sterile water, or aqueous buffer systems.

After pharmaceutical compositions have been prepared, they can be placed in an appropriate container and labeled for treatment of an indicated condition. For administration of a composition of the invention, such labeling would include amount, frequency, and method of administration.

The administration of the component of the plasminogen activating system can be repeated at least once. For example, the component of the plasminogen activating system can be administered at regular intervals, e.g., at least about once per second day, about once a day, or about twice a day, or added in wound dressings or slow-release devices which are changed as appropriate.

### Animal Models

Novel methods to screen for or evaluate candidate compounds or treatment methods that improve the healing of wounds are provided. Because of the concise structure and high similarity with skin tissue, tympanic membranes provide a unique opportunity to study the general mechanisms of wound healing. Perforations of a standardized size can be performed and the healing pattern followed by various techniques, e.g., in the otomicroscope and light microscope (Hellstrom et al., In: Fidia Research Series, Vol. 8, Liviana Press, Padova 1989). Thus, drug testing and screening for compounds increasing the wound concentration of plasminogen, as well as dosages and administration routes, can advantageously be studied in such animal models. The animal models of the invention also provide for drug screening for conditions where the healing process is slow or inhibited.

In accordance with the present invention, wild-type or plasminogen-deficient mice can be used to screen for compounds that improve wound healing and/or reduce scar formation. A compound for treatment of wounds, as well as diseases and conditions associated with ECM degradation, has been identified and verified using this model. However, also wild-type animals, as well as other gene-deficient animals could be used in the context of this model.

The animals used in these methods may be wild-type experimental animals conventionally used in the art, or may be genetically modified animals or “transgenic” animals. Transgenic mammals can be prepared by any method, including but not limited to modification of embryonic stem (ES) cells and heteronuclear injection into blast cells. Particularly preferred animal models include homozygous or heterozygous plasminogen-deficient mice (see, e.g., Ploplis et al. Circulation 92, 2585-2593 (1995)). As an example, plasminogen was identified as a compound improving wound healing and reducing scar formation as described in the Examples.

For example, a “knockout mammal” is a mammal (e.g., mouse) that contains within its genome a specific gene that has been inactivated by the method of gene targeting (see, e.g., U.S. Pat. Nos. 5,777,195 and 5,616,491). A knockout mammal includes both a heterozygote knockout (i.e., one defective allele and one wild-type allele) and a homozygous mutant. Preparation of a knockout mammal requires first introducing a nucleic acid construct that will be used to suppress expression of a particular gene into an undifferentiated cell type termed an embryonic stem cell. This cell is then injected into a mammalian embryo. A mammalian embryo with an integrated cell is then implanted into a foster mother for the duration of gestation. Zhou et al. (Genes and Development 9:2623-34 (1995)) describe PPCA knock-out mice, and the Examples provided herein describe animals in which one or both alleles of plasminogen have been knocked-out.

A “knock-in” mammal is a mammal in which an endogenous gene is substituted with a heterologous gene (Roemer et al., New Biol. 3:331-5 (1991)). Preferably, the heterologous gene is “knocked-in” to a locus of interest, either the subject of evaluation (in which case the gene may be a reporter gene; see Elegant et al., Proc. Natl. Acad. Sci. USA 95:11897 (1998)) of expression or function of a homologous gene, thereby linking the heterologous gene expression to transcription from the appropriate promoter. This can be achieved by homologous recombination, transposon (Westphal and Leder, Curr Biol 7:530 (1997)), using mutant recombination sites (Araki et al., Nucleic Acids Res 25:868 (1997)) or PCR (Zhang and Henderson, Biotechniques 25:784 (1998)). In the context of the invention, human plasminogen can be “knocked-in” instead of the CIA mouse counterpart to study the effects of a drug directed against any of these drug targets.

In another series of embodiments, transgenic animals can be created in which (i) a human plasminogen gene is stably inserted into the genome of the transgenic animal; and/or (ii) the endogenous corresponding genes are inactivated and replaced with their human counterparts (see, e.g., Coffman, Semin Nephrol. 17:404 (1997); Esther et al., Lab. Invest. 74:953 (1996); and Murakami et al., Blood Press. Suppl. 2:36 (1996)). Such animals can be treated with candidate compounds and monitored for wound healing or level/activity of plasminogen or plasmin.

Transgenic animals based on the tympanic membrane perforation model can be prepared for studying new drug targets for wound healing, or for evaluating potential drugs affecting the healing of wound diseases. Such animals provide excellent models for screening or testing drug candidates. Human plasminogen-activation “knockout” animals can be prepared to identify novel drug targets as described in the Examples, and “knock-in” mammals can be prepared for evaluating drug effects on human counterparts of the plasminogen-activation system. Both technologies permit manipulation of single units of genetic information in their natural position in a cell genome and to examine the results of that manipulation in the background of a terminally differentiated organism.

Because of the concise structure and high similarity with skin tissue, tympanic membrane perforations provide a unique opportunity to study the general mechanisms of wound healing. The tympanic membrane is an extremely standardized model that gives very well defined wounds since it has an exact and very defined composition. It has 3 layers and one of the layers are composed of 5-6 keratinocytes depth; the middle layer is a connective tissue layer composed with very thin collagen II, the mucosal inner layer is made of epithelium cells. Thereby, wound healing can be studied at a cellular level. By contrast, in the case of skin wound, it may be difficult to evaluate and standardize the depth of the wound.

### EXAMPLES

The invention is further described by means of the following examples. However, these examples are only illustrative of the invention, and in no way limits the scope and meaning of the invention. Indeed, many modifications and variations of the invention will be apparent to those skilled in the art upon reading this specification, and can be made without departing from its sprit and scope.

### Example 1

### Healing of Disrupted Tympanic Membranes in Wild-Type and Plasminogen-Deficient Mice

This Example shows that plasminogen-deficient mice had delayed and abnormal wound healing compared to wild type control siblings. Short term studies between plasminogen deficient and wild type control mice showed from as early as 6 hours after perforation performed, plasminogen deficient mice had much higher inflammatory response indicating plasminogen function as early as the inflammation stage of wound healing.

### Methods

Mice. Adult male plasminogen gene deficient and wildtype sibling mice (C57BL/6J, 8-12 week old) were typed by a chromogenic activity assay, which determines the level of plasminogen in mouse plasma (Ny et al., Endocrinology. 140(11):5030-5 (1999)) and the genotype confirmed with PCR (WO 03/20297). The mice were anesthetized and under an otomicroscope their tympanic membranes were perforated with a myringotomy lancet. The perforation occupied the upper posterior quadrant of the tympanic membrane.

Clinical evaluation of wound healing and histological preparations. Wound healing appearance was evaluated under otomicroscope at 4, 8, 36, 72, and 144 days after perforation, respectively. At each time point, mice were decapitated, their tympanic bullas were cleaned from soft tissue, opened and put into 2% glutaraldehyde in cacodylate buffer overnight. The tympanic membrane, including pars tensa and pars flaccida, was dissected, rinsed and postfixed in osmium tetroxide. After dehydration in acetone, the specimens were embedded in a epoxy resin. The plastic embedded tympanic membranes were sectioned parallel to the handle of the malleus through the perforated quadrant. The toluidine-blue stained, 0.5 micron sections were then examined in the light microscope. Ultra-thin sections, 700 nm in thickness, were also cut and contrasted with uranyl acetate and lead citrate and then studied in the electron microscope.

### Results

The healing of tympanic membrane was impaired in plasminogen deficient mice, as revealed by histological and morphological examination (see Table 2). Table 2 shows the results from the evaluation of the otomicroscopical appearance of the tympanic membrane, as a function of time after perforation. The numbers of overtly closed tympanic membranes are given in relation to the total number examined. Morphological analysis revealed the wild type mice perfectly healed, however, all the perforations in plasminogen deficient mice had totally disrupted healing pattern compared to wildtype.

TABLE 2
Healing of tympanic membrane is permanently impaired in
plasminogen deficient mice
The table shows the fraction of overtly covered tympanic
membranes in each group at each time point.

| | Time after perforation (days): | | | | | |
|---|---|---|---|---|---|---|
| | 4 | 8 | 11 | 36 | 72 | 144 |
| Plasminogen-deficient mice: | 2/24 | 6/14 | 0/6 | 1/6 | 8/30 | 0/12 |
| Wild-type mice: | 0/18 | 10/14 | 10/10 | 10/10 | 24/24 | 12/12 |

In addition, data from the healing of mice that are heterozygote for the plasminogen gene data were evaluated. These mice have 50% of the plasminogen concentration in their body fluids and they were found to have a retarded healing as compared to wild type mice. This shows that the healing process is dose-dependent and thus that administration of plasminogen to wild-type mice and humans could speed up the healing process.

### Example 2

### Tympanic Membrane Healing in Plasminogen-deficient Mice Reconstituted with Plasminogen

This Example demonstrates that by reconstitution of plasminogen in plasminogen deficient mice by systemic administration, the phenotype was converted completely back to normal wound healing.

Methods

This experiment was performed in a similar manner as Example 1, except for administration of plasminogen to one group of animals.

Reconstitution of plasminogen in plasminogen deficient mice. Human plasminogen was reconstituted in plasminogen-deficient mice by repeated intravenous injections of 1.5 mg plasminogen in 100 μl phosphate-buffered saline (PBS). The first dose was administered 12 hours prior to perforation of the tympanic membrane. Thereafter, plasminogen was administered every 24 hours throughout the duration of the experiment.

Results

Reconstitution of plasminogen in plasminogen deficient mice by i.v. injection restored the normal wound healing in plasminogen deficient mice. Table 3 shows the results from the evaluation of otomicroscopical appearance of the tympanic membrane after healing. The number of healed tympanic membranes are given in relation to the total number examined.

TABLE 3
Tympanic membrane healing in Plg deficient mice after
Plg administration.
The table shows the fraction of healed tympanic membranes
in each group at each time point.

| | Time after | | |
|---|---|---|---|
| | perforation | | |
| | (days): | | |
| | 4 | 8 | 11 |
| Plg^{-/-} mice: | 0/3 | 0/3 | 0/3 |
| Wild type mice: | 0/3 | 2/3 | 3/3 |
| Plg^{-/-} injected with plg: | 0/3 | 2/3 | 3/3 |

Thus, in plasminogen deficient mice wound healing of the tympanic membrane is abnormal, whereas healing of the tympanic membrane is restored in plasminogen deficient mice after administration of plasminogen. In addition, after 144 days, none of the plasminogen deficient mice had normally healed tympanic membranes, the healing pattern was completely aberrant in that the tympanic membrane became thick and non-transparent, with whitish cobble-stone like tissue covering the wound area. Morphology analysis revealed that the tissue filling up the wound area had a fibrin-like structure, indicating that cell migration had been hindered. Necrosis was apparent from Day 16 and onwards.

### Example 3

### Healing of Tympanic Membrane Perforations in tPA-Deficient Mice

Tympanic membranes (TM) of tPA-deficient (tPA^{-/-}) and wild-type mice were perforated at day 0, and the healing pattern was followed under otomicrocope, as described above. The results are described in Table 4. tPA-deficient mice back-crossed 6 times to C57B1/6 background were crossed once to DBA1/J background. The heterozygous litters were used for breeding. The wild-type (tPA^{+/+}) and homozygous (tPA^{-/-}) offspring from these breedings were used in the wound-healing experiments.

TABLE 4
Healing Of Tympanic Membranes In tPA-Deficient Mice.
The table shows the fraction of healed tympanic membranes in each
group at each time point.

Time after
perforation (days):

| | 4 | 6 | 8 | 12 |
|---|---|---|---|---|
| tPA^{+/+} mice: | 0/20 | 5/14 | 9/14 | 6/6 |
| tPA^{+/+} mice: | 0/24 | 4/16 | 10/16 | 8/8 |

The healing pattern of TM perforation in tPA^{-/-} mice was identical to that of wild type control mice, and the quality of healed TM tissue in tPA^{-/-} mice and wild type controls appeared identical, in that no “cobble-stone-like” tissue or fibrin deposits could be observed in the wound area. Accordingly, no significant quantitative or qualitative differences in tympanic membrane healing were observed in tPA-deficient mice as compared to wild-type, thereby indicating that tPA plays a lesser, if any, role in tympanic membrane healing.

### Example 4

### Healing of Tympanic Membrane Perforations in uPA-Deficient Mice

Tympanic membranes in wild-type and uPA-deficient (uPA^{-/-}) mice were perforated at day 0, and the healing pattern monitored by otomicrocope as described above. The results are set forth in Table 5. uPA-deficient mice backcrossed 6 times to C57B1/6 background were crossed once to DBA1/J background. The heterozygous litters were used for breeding. The wild-type (uPA^{+/+}) and homozygous (uPA^{-/-}) offspring from these breedings were used in the wound-healing experiments.

TABLE 5
Healing Of Tympanic Membranes In uPA-Deficient Mice.
The table shows the fraction of healed tympanic membranes in each
group at each time point.

Time after
perforation (days):

| | 4 | 8 | 16 | 30 |
|---|---|---|---|---|
| Wild-type mice: | 0/12 | 5/8 | 8/8 | 6/6 |
| uPA^{-/-} mice: | 0/16 | 5/12 | 7/8 | 3/4 |

This experiment showed that healing of tympanic membrane perforation in uPA-deficient mice was somewhat delayed as compared to wild type controls, and whitish and cobble-stone-like tissue, with fibrin depositions, could be observed in the perforated area after healing. These data suggest that uPA may play a role in the tympanic membrane or wound healing process, potentially affecting the clearance of fibrin depositions, although to a lesser degree than plasminogen.

### Example 5

### “Late” Administration of Plasminogen

This example shows that an impaired healing process can be restored by the administration of plasminogen several weeks after the tympanic membrane perforation was inflicted.

Methods. Plasminogen-deficient (p1g^{-/-}) mice were prepared as described above. The tympanic membranes were perforated at Day 0. On day 36 and onwards for 7 days, one group of the p1g^{-/-} mice were injected daily with 1.5 mg human plasminogen in 150 μl solution.

Results. A reduction of the abnormally accumulated extracellular matrix due to administration of plasminogen could be detected by otomicroscopy. Specifically, in the plasminogen-deficient mice that received daily administrations of human plasminogen, an inflammatory reaction started which resulted in an exudation of accumulated material from the tympanic membrane area within 2 days after the first administration. During the 7-day injection period, these mice showed greatly diminished thickness of the abnormally accumulated extracellular matrix (mainly consisting of fibrin and necrotic tissue). After this initial 7-day period, the healing pattern of some tympanic membranes resembled that of normal healing. These results indicate that plasminogen is essential for the fibrin clearance and removal of necrotic tissue.

In a similar plasminogen restoration experiment, plasminogen deficient mice received plasminogen during either days 0-3, days 4-7 or days 8-11 after the perforation. This experiment showed that plasminogen was important during all three stages of the wound healing: the inflammatory stage, the tissue formation stage and the tissue remodeling stage.

### Example 6

### Topical Application of Plasminogen on Perforated Tympanic Membranes in Wild-Type Rats

This example shows that healing of tympanic membrane perforations is improved in wild-type rats by local application of plasminogen.

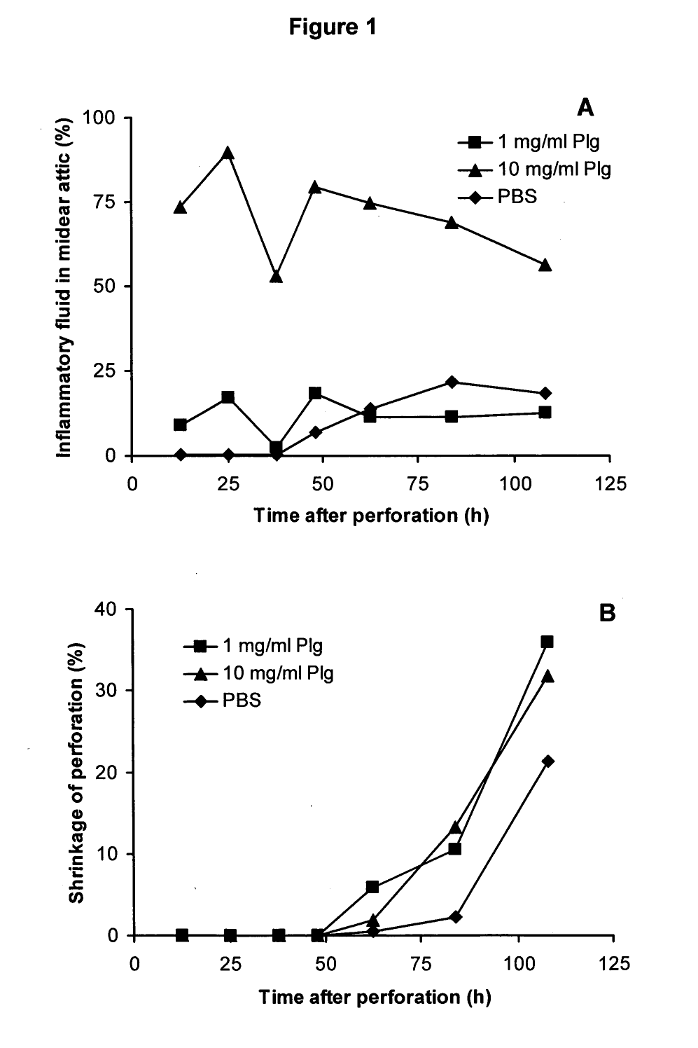

Methods. Ten tympanic membranes per animal study group were punctured. Fifty μl of PBS (control) or human plasminogen at a concentration of 1 mg/ml or 10 mg/ml was applied directly onto the tympanic membrane perforation. The addition of plasminogen or control was repeated every 24 hours, and the inflammatory reaction and healing pattern were monitored at different time points. Specifically, the accumulation of inflammatory fluid in the midear cavity was followed at the attic area (FIG. 1A), and the shrinkage of the TM perforations was followed during the first 108 hours after the perforations (FIG. 1B).

Results. The mean healing time in each group was followed and is shown in Table 6 below. The table shows the average time period for healing of a tympanic membrane perforation for each group.

TABLE 6
Improvement of Healing of Tympanic Membrane Perforations In
Wild-Type Rats by Administration of Plasminogen

| | Group: | | |
|---|---|---|---|
| | Control (PBS) | 1 mg/ml Plg | 10 mg/ml Plg |
| Time to Healing (days): | 8.1 +− 0.5 | 7.4 +− 0.6 | 6.8 +− 0.5 |

The rats that received 10 mg/ml of plasminogen had the strongest inflammatory reaction in the attic area of the mid-ear, while rats that received 1 mg/ml plasminogen displayed an almost similar inflammatory reaction as controls (FIG. 1A). The fastest healing of tympanic membranes was observed in the 10 mg/ml group, although both groups receiving plasminogen showed an improved shrinkage of the perforation during the first 108 hours as compared to controls (FIG. 1B).

### Example 7

### Characterization of Tissue Remodeling and Cell Migration Events in Plasminogen-Deficient Mice

In order to characterize the abnormal tissue remodeling and cell migration events in plasminogen-deficient (p1g^{-/-}) mice, serial immunostainings of keratin, fibrin and neutrophils were conducted on healing/healed tympanic membranes from wild-type and p1g^{-/-} mice at days 4, 8, 16, 36, 72 and 144 after perforation. The neutrophil-reactive antibody was from Cedarlane (Canada), the anti-keratin antibody was from ICN Pharmaceuticals, Inc., and the fibrinogen/fibrin-reactive antibody was from Nordic Immunology.

Results. At day 36 and onwards after the perforation, plasminogen-deficient mice had an abnormal extracellular matrix composition compared to wild type mice. In the plasminogen-deficiant mice, the perforations contained increased amounts of fibrin instead of keratin, while keratin was “kept” at the perforation border. Under otomicroscope, these fibrin deposits are seen as whitish, “cobble-stone-like” crust tissue. Large amounts of neutrophils had also infiltrated into the wounded area. Furthermore, necrotic tissue was apparent in all plasminogen-deficient mice at day 16 after perforation and onwards. The wild-type mice displayed a healed wound area identical to normal wild-type controls.

These data suggest that plasminogen, either directly or via the formation of plasmin, is important for preventing or reducing fibrin depositions, promoting keratin layer formation, as well as removing necrotic tissue.

### Example 8

### Healing Effect of Plasminogen on Burn Wounds in Rats

This experiment was to investigate if plasminogen can speed up the healing of burn wounds in rats.

Methods. The burn wounds were established by inducing a 1 cm diameter circle of burn with 100° C. for 8 seconds. Thereafter, local injections of human plasminogen (10 μg/μ1) of 40 μl in each of the two sites daily from days 3 to 20. Wound sizes were documented by photographing.

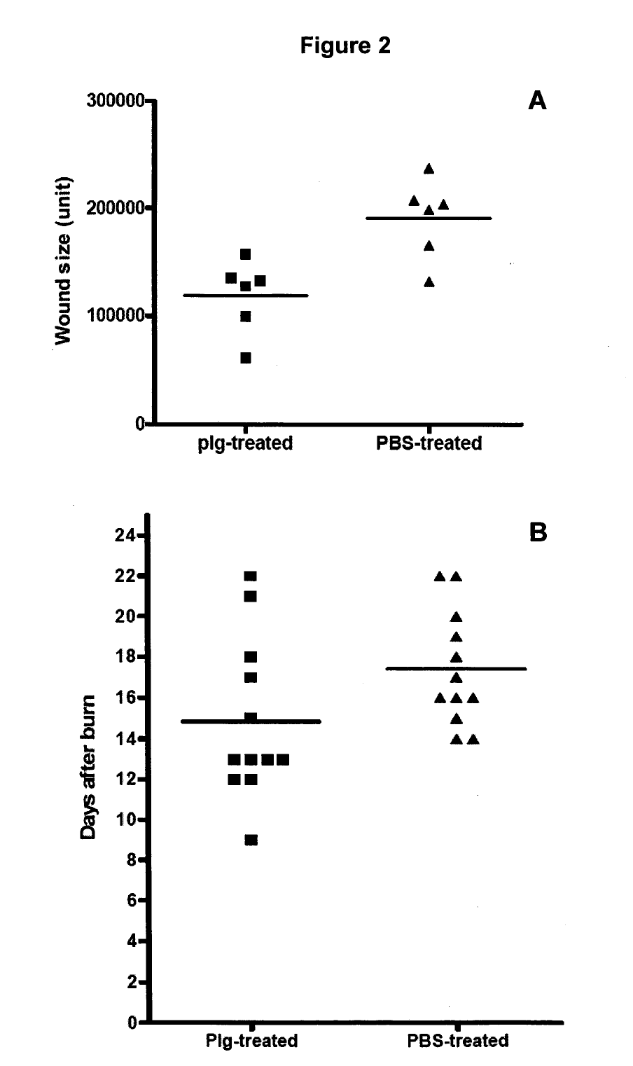

Results. As shown in FIG. 2A, 10 days after treatment, the average wound size of Plasminogen-treated group was 37% less than it in PBS-treated group (p=0.0056). The duration of the scab before it fell off in plasminogen-treated rats were 2.5 days earlier than it in PBS-treated rats (p=0.04) (FIG. 2B).

Morphological analysis also confirmed that the plasminogen-treated group in general showed better healing than the PBS-treated group. These data demonstrate that plasminogen improves burn wound healing in rats.

### Example 9

### Healing Effect of Plasminogen in Chronic Incisional Wounds

This experiment was to investigate whether plasminogen can improve the healing of chronic incisional wounds in rats

Methods. The chronic incisional wounds were induced by incising a 1.5 cm long on the back of rats at day 0 and topically applied of 100 μl 2% hydrocortisone from day 0 to day 5. From day 9 to day 16, rats were injected with 25 μl of human plasminogen (10 μg/μl) or PBS at two sides of the incisions each. Photos were taken daily to document the healing process. At day 23, rats were sacrificed and samples were taken for morphological analysis.

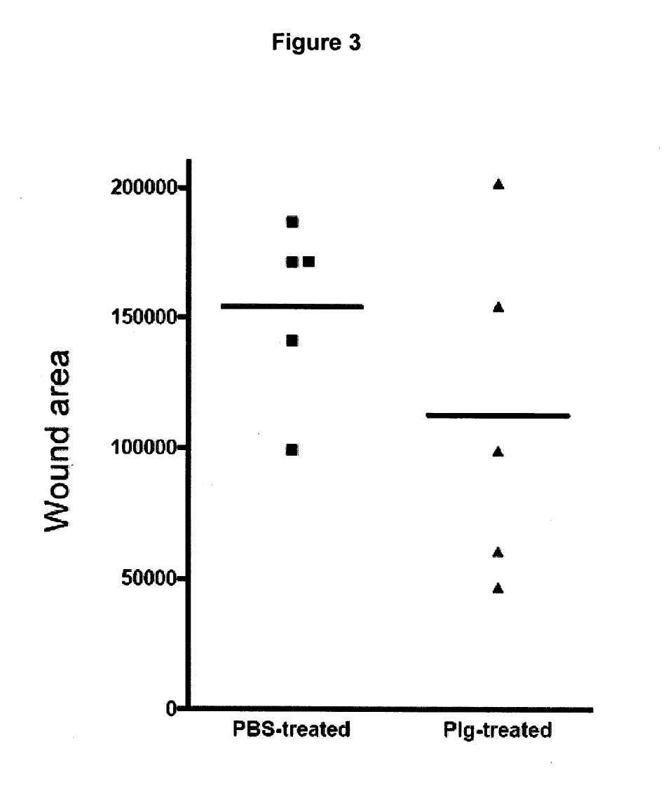

Results. Macroscopic appearance at 8 days after local injections of PBS or plasminogen suggested that plasminogen improved the healing of the chronic wounds. When no hydrocortisone was used, all of the three samples have basically healed their wounds. Further quantatitive analysis of the areas of the wounded areas indicated a 27% improvement of healing by plasminogen local treatment. (FIG. 3). These data clearly demonstrated that plasminogen improves the healing of chronic incisional wounds in rats.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that values are approximate, and are provided for description.

Numerous references, including patents, patent applications, figures, database references, and various publications, are cited and discussed in the description of this invention. All of these references are incorporated herein by reference in their entirety and to the same extent as if each reference was individually incorporated by reference.

1. A method of promoting healing of a tympanic membrane perforation, which comprises administering to a subject in need of such treatment a composition containing an effective amount of a component of the plasminogen activating system for promoting healing of the tympanic membrane perforation.

2. The method of claim 1, wherein the subject is a human, and the component of the plasminogen activating system is selected from mini-plasminogen, mini-plasmin, micro-plasminogen, micro-plasmin, delta-plasminogen, and delta-plasmin.

3. The method of claim 1, wherein the composition further comprises a pharmaceutically acceptable carrier.

4. The method of claim 3, wherein the composition is selected from a member selected from the group consisting of an aqueous solution, a gel, a lotion, a balm, a powder, a paste, a bandage, or a wound dressing.

5. The method of claim 4, wherein the composition is administered by a spray.

6. The method of claim 4, wherein the administering is selected from topical and systemic administration.

7. The method of claim 6, wherein the administering is topical administration.

8. The method of claim 1, wherein promoting healing is selected from accelerating healing of the perforation, reducing necrotic tissue, and reducing the formation of scar tissue.

9. The method of claim 1, wherein the administering is repeated at least once.

10. The method of claim 9, wherein the administering is repeated at least every day.

11. A method of reducing scar formation from a healing wound, which comprises administering to a subject in need of such treatment a composition containing an effective amount of a component of the plasminogen activating system for reducing scar formation.

12. The method of claim 11, wherein the component of the plasminogen activating system reduces fibrin deposition.

13. The method of claim 11, wherein the subject is a human, and the component of the plasminogen activating system is selected from mini-plasminogen, mini-plasmin, micro-plasminogen, micro-plasmin, delta-plasminogen, and delta-plasmin.

14. A method of accelerating wound healing, which comprises administering to a subject in need of such treatment a composition containing an effective amount of a component of the plasminogen activating system to promote healing of the wound.

15. The method of claim 14, wherein the wound is a chronic wound.

16. The method of claim 14, wherein the subject is a human, and the component of the plasminogen activating system is selected from mini-plasminogen, mini-plasmin, micro-plasminogen, micro-plasmin, delta-plasminogen, and delta-plasmin.

17. A method of reducing necrotic tissue formation in a healing wound, which comprises administering to a subject in need of such treatment a composition containing an effective amount of a component of the plasminogen activating system for reducing necrotic tissue formation.

18. The method of claim 17, wherein the component of the plasminogen activating system reduces fibrin deposition.

19. The method of claim 17, wherein the subject is a human, and the component of the plasminogen activating system is selected from mini-plasminogen, mini-plasmin, micro-plasminogen, micro-plasmin, delta-plasminogen, and delta-plasmin.
